(19) United States
(12) Patent Application Publication
(10) Pub. No.: US 2010/0145171 A1
(43) Pub. Date: Jun. 10, 2010

(54) APPARATUS FOR MEASURING MOTION NOISE ROBUST PULSE WAVE AND METHOD THEREOF

(75) Inventors: Chankyu PARK, Daejeon-city (KR); Joochan Sohn, Daejeon-city (KR); Jaehong Kim, Daejeon-city (KR); Hyeonsung Cho, Daejeon-city (KR)

Correspondence Address: STAAS & HALSEY LLP, SUITE 700, 1201 NEW YORK AVENUE, N.W., WASHINGTON, DC, 20005, US

(73) Assignee: ELECTRONICS AND TELECOMMUNICATIONS RESEARCH INSTITUTE, Daejeon-city (KR)

(21) Appl. No.: 12/630,221

(22) Filed: Dec. 3, 2009

(30) Foreign Application Priority Data
Dec. 5, 2008 (KR) 10-2008-0123489
Oct. 30, 2009 (KR) 10-2009-0104129

(51) Int. Cl. A61B 5/02; A61B 5/1455

(52) U.S. Cl. 600/324; 600/336

(57) ABSTRACT

Provided are a method of measuring the pulse wave at the back of a wrist, etc. where measurement of the pulse wave is difficult so as to prevent a user to feel inconvenience in a mobile environment and a method of detecting the pulse wave at a write portion or at the back of the wrist which has comparatively weak restraint force in a human body by recovering an original signal with comparatively minimum errors so as to be robust to motion noise according to motion of the wrist.

### RELATED APPLICATIONS

The present application claims priority to Korean Patent Application Serial Number 10-2008-0123489, filed on Dec. 5, 2008 and Korean Patent Application Serial Number 10-2009-0104129, filed on Oct. 30, 2009, the entirety of which are hereby incorporated by reference.

### BACKGROUND OF THE INVENTION

1. Field of the Invention

The present invention relates to an apparatus for measuring a motion noise robust pulse wave and a method thereof, and more particularly, to an apparatus for measuring a motion noise robust pulse wave at the back of a wrist, i.e., that part of the wrist which extends into the back of the hand, through an adaptive signal processing technique by estimating a motion component using an acceleration sensor and a method thereof.

2. Description of the Related Art

A pulse wave measuring technology as an important field that has been researched in a bio-signal processing technology field is generally used to extract a weak pulse wave bio-signal mixed in noise.

The pulse wave measuring technology is largely classified into an invasive type or a non-invasive type. The invasive type is a method of directly measuring the bio-signal in a human body and the non-invasive type is a method of indirectly measuring the bio-signal in the human body. The non-invasive type is primarily used for extracting a health index.

The non-invasive type generally measures the bio-signal by using a piezoelectric device and an optical sensor. The measurement method using the optical sensor measures the pulse wave through variations of absorption rate by using near-infrared rays output from a near-infrared LED which is the optical sensor.

In case of a principle measuring the pulse wave by using the optical sensor, volume change rate of blood that flows in a blood vessel of a radial artery or a finger artery is expressed as variations of absorption rate of the near-infrared rays by contraction/relaxation operations of heart beat by using a modified principle when the Beer-Lambert rule acquiring absorption rate of light in a predetermined medium is applied to human tissue and a near-infrared ray photo detector as a receiver acquires near-infrared rays that are transmitted into or reflected on the blood vessel.

### SUMMARY OF THE INVENTION

In measuring a pulse wave in a u-health care field based on a ubiquitous environment, it is most important to acquire user's accurate bio-signal information. However, in order to measure the pulse wave in a portable environment, there are various problems depending on measured parts and characteristics.

For the portable environment, an apparatus for measuring the pulse wave should be able to be worn in a part of a human body to be measured and in a portable environment which is not stable such as a patient in a hospital, a pulse wave signal is distorted due to movement of the measurement part, such that it cannot be used as health index data.

In recent years, in order to solve the motion noise, a research for minimizing the motion noise has been in progress by adopting techniques such as adaptive signal processing, wavelet signal processing, morphological signal processing, etc. even in the existing medical equipment.

In order to solve the above-mentioned problem, an object of the present invention is to provide an apparatus for measuring a motion noise robust pulse wave and a method thereof that can measure a pulse wave at the back of a wrist, etc. where measurement of the pulse wave is difficult so as to prevent a user to feel inconvenience in a mobile environment.

Further, another object of the present invention is to provide an apparatus for measuring a motion noise robust pulse wave and a method thereof that are robust to motion noise according to motion of a wrist by recovering an original signal with comparatively minimum errors.

In order to achieve the above-mentioned object, an apparatus for measuring a motion noise robust pulse wave according to an aspect of the present invention includes: a PPG sensor that is disposed at the back of a wrist of an examinee to detect a pulse wave signal from a signal output to the back of the wrist of the examinee; an acceleration sensor that detects motion of the examinee at a portion where the PPG sensor is mounted; and a signal preprocessor that estimates a motion noise model corresponding to the motion of the examinee by predicting the motion of the examinee on the basis of an acceleration signal detected by the acceleration sensor and removes motion noise for the pulse wave signal from the motion noise model.

The signal preprocessor includes a signal acquiring unit that samples the pulse wave signal and the acceleration signal by a predetermined unit; and a noise remover that removes noise of the pulse wave signal sampled by the signal acquiring unit by using a high-pass filter.

The motion noise model is a unique motion noise model and the signal preproessor acquires motion noise having high correlation on the basis of the unique motion noise model.

The signal preprocessor estimates the unique motion noise model by using an AR (Auto Regressive) model estimator.

The signal preprocessor outputs the motion noise signal having high correlation with unique motion noise of the examinee by filtering the unique motion noise model and the acceleration signal detected by the acceleration sensor. At this time, the signal preprocessor uses an FIR (Finite Impulse Response) filter.

The signal preprocessor removes the motion noise of the pulse wave by using the motion noise signal having high correlation with the unique motion noise of the examinee.

The apparatus further includes a communication unit that transmits the pulse wave signal from which the motion noise is removed to an external host server.

The PPG sensor includes a light emitting unit that outputs an optical signal to the back of the wrist of the examinee; and a light receiving unit that receives the optical signal output by the light emitting unit.

The acceleration sensor includes a 3-axis acceleration sensor and detects an acceleration component of each axis in accordance with the motion of the examinee by using the 3-axis acceleration sensor.

Meanwhile, in order to achieve the above-mentioned object, a method for measuring a motion noise robust pulse wave according to another aspect of the present invention includes: detecting a pulse wave signal from a PPG sensor disposed at the back of a wrist of an examinee; detecting motion of the examinee at the portion where the PPG sensor is mounted by using an acceleration sensor; estimating a motion noise model corresponding to the motion of the examinee on the basis of the motion information by the examinee and the acceleration signal detected by the acceleration sensor; and removing the motion noise for the pulse wave signal from the motion noise model.

The method further includes acquiring motion noise having high correlation with the unique motion noise of the examinee on the basis of the estimated motion noise model and the acceleration signal detected by the acceleration sensor.

In the removing the motion noise, the motion noise of the pulse wave signal is removed by using the motion noise signal having high correlation with the unique motion noise of the examinee.

In the removing the motion noise, the motion noise of the pulse wave signal is repetitively removed by using an adaptive filter

The method further includes transmitting the pulse wave from which the motion noise is removed to an external host server.

According to an embodiment of the present invention, in order to solve the above-mentioned two problems, there are provided a method of measuring the pulse wave at the back of a wrist, etc. where measurement of the pulse wave is difficult so as to prevent a user to feel inconvenience in a mobile environment and it is possible to detect the pulse wave at a write portion or at the back of the wrist which has comparatively weak restraint force in a human body by recovering an original signal with comparatively minimum errors so as to be robust to motion noise according to motion of the wrist.

Further, it is possible to remove the motion noise from a distorted pulse wave measured by adding the motion noise by using a statistical model, an acceleration sensor, and an adaptive signal processing technology.

Meanwhile, in the present invention, a watch-shape wrist-wearable apparatus is provided so as to be used in a portable environment so as to prevent a user to feel inconvenience.

Further, the present invention has an advantage of checking and managing a personal health index using the pulse wave in a PC, a notebook, a mobile phone, a PDA, and other mobile apparatuses supporting Bluetooth/Zigbee.

In addition, with an increase in concern about health, the present invention may be used as a health measurement means for adults or old men and an emergency precursor situation detection function and when contents for stress, concentration, sleep quality, etc. through HRV analysis are provided to a host device, the present invention may be used as more various purposes, that is, for a game, education, wellbeing contents and in addition, when the wireless protocol such as Zigbee is used, since data of a plurality of wearable apparatuses in groups such as a hospital, a senior center, and a silver town can be collected and processed, the present invention may be used as a basic apparatus in a ubiquitous-based health care field.

### BRIEF DESCRIPTION OF THE DRAWINGS

FIG. 1 shows a configuration of a system to which an apparatus for measuring a motion pulse wave is applied according to the present invention;

FIG. 2 is a block diagram reference for describing a configuration of an apparatus for measuring a pulse wave according to the present invention;

FIG. 3 is a block diagram referenced for describing a configuration of a host device according to the present invention;

FIG. 4 is a diagram showing a structure of a pulse wave measuring apparatus according to the present invention;

FIGS. 5A and 5B are exemplary diagrams referenced for describing an operation of an apparatus for measuring a pulse wave according to the present invention; and

FIG. 6 is a diagram referenced for describing an operation to remove motion noise in an apparatus for measuring a pulse wave according to the present invention.

### DETAILED DESCRIPTION OF THE PREFERRED EMBODIMENTS

Hereinafter, embodiments of the present invention will be described with reference to the accompanying drawings.

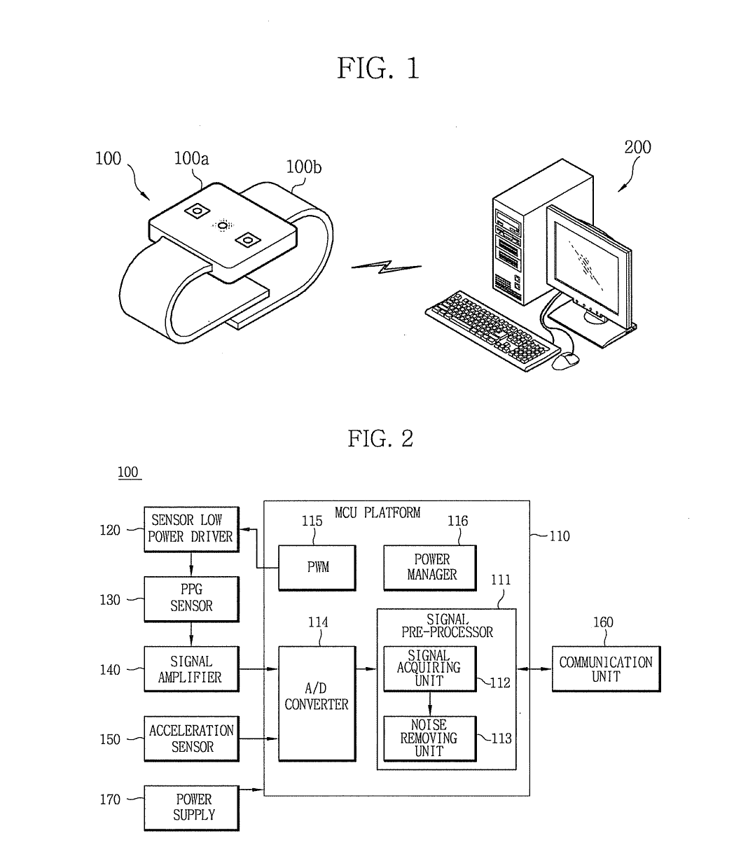

FIG. 1 shows a configuration of a system to which an apparatus for measuring a motion noise robust pulse wave is applied according to the present invention.

The pulse wave measuring apparatus 100 according to the present invention has a communication protocol for using Bluetooth or Zigbee. When the pulse wave measuring apparatus 100 measures a pulse wave from an examinee, the pulse wave measuring apparatus 100 can transmit measured data to a PC, a robot, and other medical equipments which is a host device 200 by using a wireless communication technology.

At this time, the pulse wave measuring apparatus 100 according to the present invention includes a body 100a for measuring the pulse wave of the examinee and a wearing means 100b for wearing the body 100a on a wrist. The wearing means 100b can be implemented in a band form and may be implemented in a form such as a wrist watch or a wristlet.

Therefore, referring to FIG. 2, the configuration of the pulse wave measuring apparatus will be described in more detail.

FIG. 2 is a block diagram showing a detailed configuration of an apparatus for measuring a motion noise robust pulse wave according to the present invention. Referring to FIG. 2, the pulse wave measuring apparatus 100 according to the present invention includes an MCU platform 110, a sensor low power driver 120, a PPG sensor 130, a signal amplifier 140, an acceleration sensor 150, a communication unit 160, and a power supply 170.

The MCU platform 110 includes a signal preprocessor 111, an A/D converter 114, a PWM 115, and a power manager 116.

The sensor low power driver 120 outputs a driving signal for driving the PPG sensor to the PPG sensor 130.

The PPG sensor 130 is disposed at the back of the wrist of the examinee. At this time, the PPG sensor 130 includes a light emitting unit (not shown) outputting an optical signal to the back of the wrist for measuring the pulse wave and a receiver (not shown) receiving the optical signal reflected on the wrist, a blood vessel, etc. of the examinee.

At this time, the PPG sensor 130 performs an operation of measuring the pulse wave according to the driving signal from the sensor low power driver 120. The PPG sensor 130 measures a pulse wave signal from the examinee and transfers the measured pulse wave signal to the signal amplifier 140.

Further, the signal amplifier 140 amplifies the pulse wave signal measured by the PPG sensor to a signal having a predetermined level and thereafter, transfers the amplified signal to the A/D converter 114 of the MCU platform.

Meanwhile, the acceleration sensor 150 has a sensor for measuring motion of the wrist or a hand of the examinee. At this time, the sensor of the acceleration sensor 150 may correspond to a gravity acceleration sensor, an angular velocity sensor, or the like. The acceleration sensor 150 transfers measured motion data to the A/D converter 114 of the MCU platform.

The motion data measured by the acceleration sensor 150 is used to compensate the motion noise in the pulse wave signal afterwards.

The A/D converter 114 converts an analog pulse wave signal inputted from the signal amplifier 140 into a digital signal and outputs the converted signal to the signal preprocessor 111. Similarly, the A/D converter 114 converts the analog signal transferred from the acceleration sensor 150 into the digital signal and outputs the converted signal to the signal preprocessor 111.

The signal preprocessor 111 serves to remove noise by preprocessing signals measured by sensors of the PPG sensor 130 and the acceleration sensor 150. At this time, the signal preprocessor 111 includes a signal acquiring unit 112 and a noise removing unit 113.

The signal acquiring unit 112 samples the pulse wave at a sampling speed having a range of 100 to 1000 pulse waves per second and the acceleration signal at a speed of 100 signals per second while quantization in the range of 10 bits to 12 bits.

The noise removing unit 113 removes optical noise, electrical noise, etc. of the pulse wave signal sampled by the signal acquiring unit 112 by using a low-pass filter. Meanwhile, the noise remover 113 removes breath noise, a direct current component, or the like of the pulse signal sampled by the signal acquiring unit 112 by using a high-pass filter.

Herein, a degree of each filter is 4, and a cutoff frequency is 1.5 Hz and 0.5 Hz and uses a butter worth type IIR filter.

Meanwhile, the noise remover 113 removes an offset voltage of the acceleration signal and removes high-frequency noise of the acceleration signal by using a smoothing filter.

Lastly, the signal preprocessor 111 serializes the pulse wave signal and the acceleration signal without noise and the serialized signals to the communication unit 160.

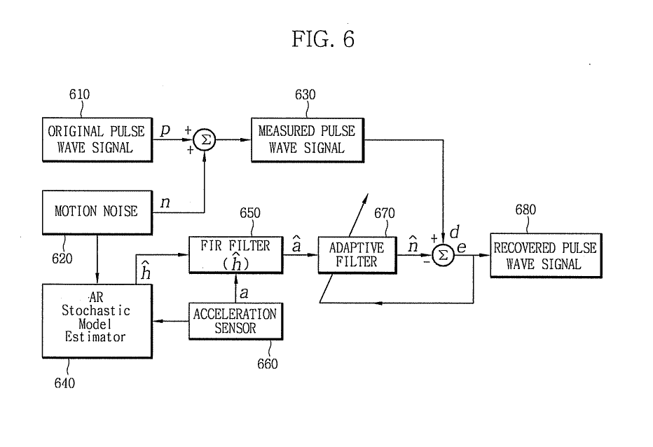

An operation of the signal preprocessor 111 will be described in detail with reference to FIG. 6.

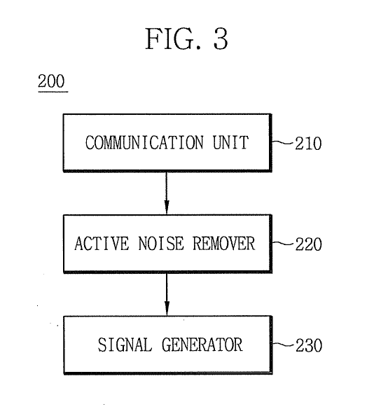

FIG. 3 is a block diagram illustrating a configuration of a host device according to the present invention.

Referring to FIG. 3, the host device 200 includes a communication unit 210, an active noise remover 220, and a signal generator 230.

The communication unit 210 receives the signal transmitted from the pulse wave measuring apparatus 100.

The active noise remover 220 removes the motion noise in an active noise removing scheme by using the pulse wave signal and the acceleration signal received through the communication unit 210.

The motion noise is removed by the active noise remover 220, such that the signal generator 230 stores the finally recovered pulse wave signal, and generates a PP signal which is an interval between peak points of pulsation from the recovered pulsation signal and provides the generated PP signal as basic data for HRV analysis.

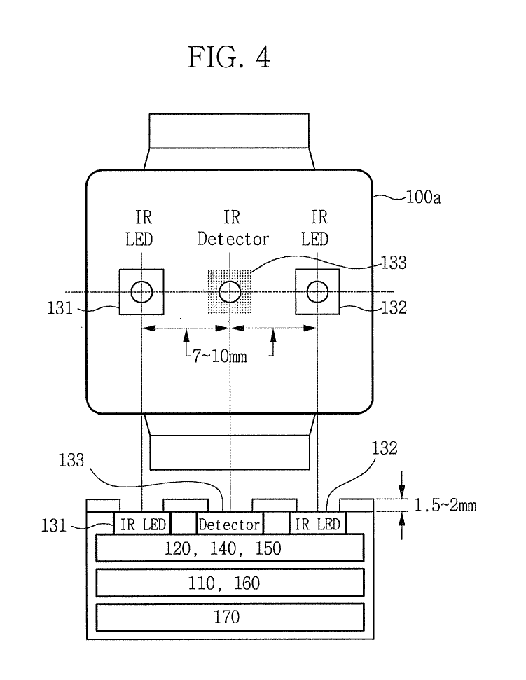

FIG. 4 is a diagram showing a structure of a pulse wave measuring apparatus according to the present invention.

Referring to FIG. 4, in the pulse wave measuring apparatus 100 according to the present invention, the PPG sensor 130 includes two light emitting devices for emitting near-infrared rays to the back of the wrist of the examinee. In the embodiment of FIG. 4, a case in which two light emitting devices are infrared ray (IR) LEDs 131 and 132 will be described as an example.

Since two IR LEDs 131 and 132 are driven by a modulated pulse to disable two IR LEDs 131 and 132 to be driven during a time interval when sampling is not performed, two IR LEDs 131 and 132 are not consecutively driven.

Further, the PPG sensor 130 further includes a light detection device that detects the near-infrared rays output by two IR LEDs 131 and 132. In the embodiment of FIG. 4, a case in which the light detection device is an IR detector 133 will be described as an example.

The IR detector 133 fully reacts to a sampling speed by appropriately adjusting a duty cycle in association with a cycle sampling an inputted signal.

Herein, two IR LEDs 131 and 132 are disposed at both sides around the IR detector 133. This is to support a wrist structure wider than a finger and find a flow of an artery at a deep location because various bodily tissues, in particular, a carpal of the wrist has a more complicated structure than distal ends of the finger and the artery is positioned deep in the wrist.

At this time, when two IR LEDs 131 and 132 and the IR detector 133 are close to each other, the IR detector 133 can directly absorb light output from the IR LEDs 131 and 132 as well as light reflected on the wrist after the light output from the IR LEDs 131 and 132 absorbs in the wrist. Therefore, two IR LEDs 131 and 132 and the IR detector 133 are disposed spaced from each other by a predetermined interval. Preferably, they are disposed spaced from each other by an interval of 7 to 10 mm.

Further, when two IR LEDs 131 and 132 and the IR detector 133 are mounted in the body 100a of the pulse wave measuring apparatus 100, they are mounted to be inserted into the body 100a rather than the surface of the body 100a. Preferably, they are mounted to be inserted inside by 1.5 to 2 mm.

In this case, it is possible to prevent the optical signals output from the two IR LEDs 131 and 132 from absorbing directly in the IR detector 133 and a body contact surface and the two IR LEDs 131 and 132 and the IR detector 133 are spaced from each other to thereby reduce motion noise generated during contact of a body.

Further, in the pulse wave measuring apparatus 100 according to the present invention, the sensor low power driver 120 that applies the driving signal to the PPG sensor 130 and the acceleration sensor for measuring motion of the examinee are disposed below the PPG sensor 130 and the MCU platform 110 is disposed.

Further, the power supply 170 including the battery is disposed below the sensor low power driver 120, the acceleration sensor, and the MCU platform 110 and supplies power to the pulse wave measuring apparatus 100. At this time, the power supply 170 has a charge circuit to supply power with a lithium ion battery (3.3 v). In addition, the power supply 170 is supported with a standby mode to be driven at low power.

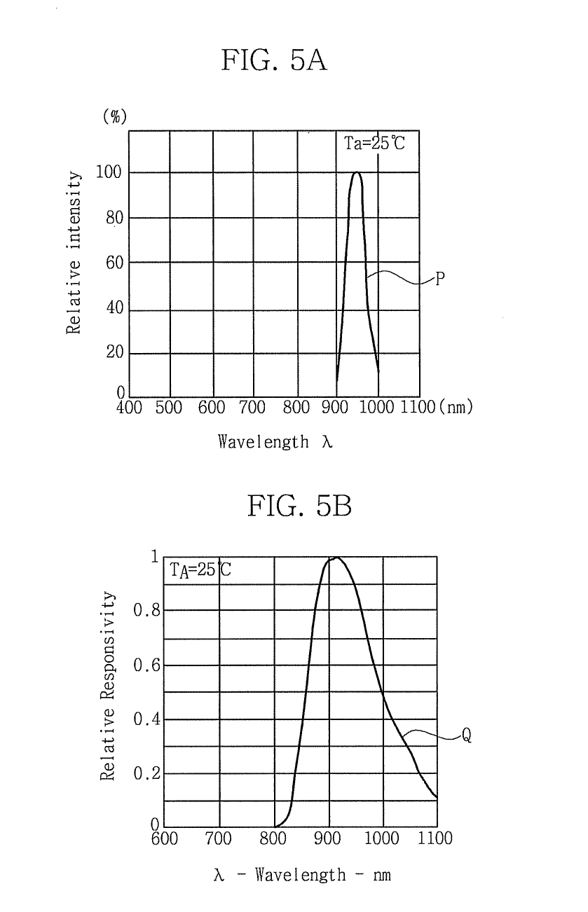

FIGS. 5A and 5B are exemplary diagrams showing a characteristic curve the IR LED and the IR detector shown in FIG. 4.

First, FIG. 5A is a graph showing an output characteristic of the IR LED.

As shown in FIG. 5A, the IR LEDs 131 and 132 output the near-infrared rays which are light having a wavelength of 940 nm. At this time, the IR LEDs 131 and 132 should have power strength such as ‘P’ in FIG. 5A.

In particular, in the pulse wave measuring apparatus 100 according to the present invention, since the IR LEDs 131 and 132 are limited within a range of 900 nm to 1000 nm, it is possible to measure the pulse wave at the back of the wrist of the examinee only by outputting the near-infrared rays.

FIG. 5B is a graph showing a response characteristic of an IR detector.

When a spread degree of a response curve is wide, light beams of other bands are absorbed in the IR detector 133 to generate noise, such that the IR detector 133 uses a sensor that well reacts in a wavelength of 800 nm to 1000 nm as shown in a response curve ‘Q’ in FIG. 5B.

Hereinafter, Equation 1 shows a case in which the Beer-Lambert rule is applied to body tissues.

$${I}_{o} ( t ) = {I}_{i} ( t ) \cdot exp ( - {\sum }_{k = 1}^{n} {ɛ}_{λ , k} {c}_{k} ( t ) {d}_{k} ( t ) ) \qquad [ Equation 1 ]$$

Herein, Ii(t) represents the intensity of inputted light and Io(t) represents the intensity of light transmitted and output to the body tissue. Further, ε_{λ,k} represents an absorption coefficient for each medium, Ck(t) represents concentration, and dₖ(t) represents a distance of each medium.

As shown in Equation 1, in case of Io(t), the intensity of the light transmitted and output from the body tissues, the intensity of the inputted light varies according to three variation factors such as the absorption coefficient for each medium, the concentration, and the distance between media.

That is, since the power intensity varies depending on the distance between the media, the concentration, and the absorption coefficient with respect to light having one wavelength, the intensity of the light may remarkably vary at a portion having a complicated body tissue such as the wrist portion.

Meanwhile, in case of using sensors of which curves of an output characteristic and a response characteristic spread, since the Beer-Lambert rule of Equation 1 should consider light beams of several wavelength bands, the Beer-Lambert rule should be modified as shown in Equation 2.

$${I}_{o} ( t ) = {\sum }_{j} {I}_{j} ( t ) \cdot exp ( - {\sum }_{k = 1}^{n} {ɛ}_{{λ}_{j} , k} {c}_{k} ( t ) {d}_{k} ( t ) ) \qquad [ Equation 2 ]$$

That is, since incident light beams of different wavelength bands are mixed to influence the power due to different absorption rate of light for each wavelength band, it is important to have characteristics of a comparatively narrow wavelength band in a wrist tissue made of a complicated medium.

FIG. 6 is an exemplary diagram referenced to describe an operation of removing motion noise in a signal preprocessor according to the present invention and shows an adaptive filter structure for an active noise remover for removing the motion noise in a distorted pulse wave signal measured by adding the motion noise.

Referring to FIG. 6, in case where there is no motion noise, a pure pulse wave signal P is generated and in case where motion is generated by the examinee, a signal adding a unique motion noise component n to the pure pulse wave P is generated.

Accordingly, a signal actually measured by the PPG sensor 130 is d=p+n, a signal adding the unique motion noise component n to the pure pulse wave signal P, which is measured.

At this time, the value of the unique noise component n cannot be accurately found. If a signal approximately having a high correlation with n can be acquired, the unique noise n included the original signal can be reduced by an error of a minimum mean square by using an active noise remover (ANC) structure in an adaptive filter.

In the pulse wave measuring apparatus 100 according to the present invention, a model of the unique motion noise is estimated by using an AR model estimator and noise having a high correlation is acquired by calculating a transfer function ĥ of the estimated model.

Meanwhile, the motion of the wrist portion acquires not the pulse wave component but an acceleration component of each axis from a 3-axis acceleration sensor of the acceleration sensor 150. At this time, the acceleration sensor transfers an acceleration signal a to an FIR filter so that the acceleration signal has a correlation with a signal distorting the pulse wave signal.

According to another embodiment, in case of using a cuff of a blood pressure meter, the AR estimator estimates a value of a pulse wave component caused by approximate unique motion noise generated through motion of the wrist after the pulse wave component in the wrist is removed.

Accordingly, the FIR filter outputs a signal a having a correlation with the unique motion noise n by filtering the transfer function ĥ and the acceleration signal a. At this time, the signal â serves as an input signal of the active noise remover.

Thereafter, the signal preprocessor 111 acquires a finally recovered signal from which final motion noise is removed to some extent according to an adaptive filter's own function.

As described above, although an apparatus for measuring a motion noise robust pulse wave and a method thereof according to the present invention have been described with reference to the accompanying drawings, the present invention is not limited by the embodiments and drawings disclosed in the present invention and may be applied with the scope if which the spirit is protected.

What is claimed is:

1. An apparatus for measuring a motion noise robust pulse wave, comprising:
a PPG sensor that is disposed at the back of a wrist of an examinee to detect a pulse wave signal from a signal output to the back of the wrist of the examinee;
an acceleration sensor that detects motion of the examinee at a portion where the PPG sensor is mounted; and
a signal preprocessor that estimates a motion noise model corresponding to the motion of the examinee by predicting the motion of the examinee on the basis of an acceleration signal detected by the acceleration sensor and removes motion noise for the pulse wave signal from the motion noise model.

2. The apparatus for measuring a motion noise robust pulse wave according to claim 1, wherein the signal preprocessor includes:
a signal acquiring unit that samples the pulse wave signal and the acceleration signal by a predetermined unit; and
a noise remover that removes noise of the pulse wave signal sampled by the signal acquiring unit by using a high-pass filter.

3. The apparatus for measuring a motion noise robust pulse wave according to claim 1, wherein the motion noise model is a unique motion noise model and the signal preproessor acquires motion noise having high correlation on the basis of the unique motion noise model.

4. The apparatus for measuring a motion noise robust pulse wave according to claim 3, wherein the signal preprocessor estimates the unique motion noise model by using an AR (Auto Regressive) model estimator.

5. The apparatus for measuring a motion noise robust pulse wave according to claim 3, wherein the signal preprocessor outputs the motion noise signal having high correlation with unique motion noise of the examinee by filtering the unique motion noise model and the acceleration signal detected by the acceleration sensor.

6. The apparatus for measuring a motion noise robust pulse wave according to claim 5, wherein the signal preprocessor uses an FIR (Finite Impulse Response) filter.

7. The apparatus for measuring a motion noise robust pulse wave according to claim 5, wherein the signal preprocessor removes the motion noise of the pulse wave by using the motion noise signal having high correlation with the unique motion noise of the examinee.

8. The apparatus for measuring a motion noise robust pulse wave according to claim 1, further comprising:
a communication unit that transmits the pulse wave signal from which the motion noise is removed to an external host server.

9. The apparatus for measuring a motion noise robust pulse wave according to claim 1, wherein the PPG sensor includes:
a light emitting unit that outputs an optical signal to the back of the wrist of the examinee; and
a light receiving unit that receives the optical signal output by the light emitting unit.

10. The apparatus for measuring a motion noise robust pulse wave according to claim 1, wherein the acceleration sensor includes a 3-axis acceleration sensor and detects an acceleration component of each axis in accordance with the motion of the examinee by using the 3-axis acceleration sensor.

11. A method for measuring a motion noise robust pulse wave, comprising:
detecting a pulse wave signal from a PPG sensor disposed at the back of a wrist of an examinee;
detecting motion of the examinee at the portion where the PPG sensor is mounted by using an acceleration sensor;
estimating a motion noise model corresponding to the motion of the examinee on the basis of the motion information by the examinee and the acceleration signal detected by the acceleration sensor; and
removing the motion noise for the pulse wave signal from the motion noise model.

12. The method for measuring a motion noise robust pulse wave according to claim 11, further comprising:
acquiring motion noise having high correlation with the unique motion noise of the examinee on the basis of the estimated motion noise model and the acceleration signal detected by the acceleration sensor.

13. The method for measuring a motion noise robust pulse wave according to claim 12, wherein in the removing the motion noise, the motion noise of the pulse wave signal is removed by using the motion noise signal having high correlation with the unique motion noise of the examinee.

14. The method for measuring a motion noise robust pulse wave according to claim 11, wherein in the removing the motion noise, the motion noise of the pulse wave signal is repetitively removed by using an adaptive filter.

15. The method for measuring a motion noise robust pulse wave according to claim 11, further comprising:
transmitting the pulse wave signal from which the motion noise is removed to an external host server.
